(19) United States
(12) Patent Application Publication
(10) Pub. No.: US 2015/0144139 A1
(43) Pub. Date: May 28, 2015

(54) PATIENT INTERFACE DEVICE HAVING A RESIDUAL LEAK SEAL CUSHION

(71) Applicant: Harold Allen Lockhart, Mt Pleasant, PA (US)

(72) Inventor: Harold Allen Lockhart, Mt Pleasant, PA (US)

(73) Assignee: KONINKLIJKE PHILIPS N.V., EINDHOVEN (NL)

(21) Appl. No.: 14/345,420

(22) PCT Filed: Aug. 10, 2012
(86) PCT No.: PCT/IB2012/054685

§ 371 Date: Mar. 18, 2014

Related U.S. Application Data
(60) Provisional application No. 61/536,697, filed on Sep. 20, 2011.

(51) Int. Cl. A61M 16/06; A61M 16/08

(52) U.S. Cl. CPC A61M 16/0622; A61M 16/0666; A61M 16/0816
USPC 128/205.25; 128/206.24

(57) ABSTRACT

A patient interface device includes a fluid coupling device, a frame member having a connecting portion defining a first opening, and a cushion member. The cushion member has a main body portion, a coupling portion attached thereto, and an inwardly extending sealing member defining a second opening in the cushion member. The coupling portion is attached to a first side of the connecting portion so that at least a portion of the sealing member extends over the first opening. The fluid coupling device is attached to a second side of the connecting portion so that the outlet end of the fluid coupling device extends through the first opening and a terminal portion of the outlet end engages the sealing member and creates an airflow seal at a joint connection between the fluid coupling conduit and the frame member.

### CROSS-REFERENCE TO RELATED APPLICATIONS

This patent application claims the priority benefit under 35 U.S.C. §119(e) of U.S. Provisional Application No. 61/536,697 filed on Sep. 20, 2011, the contents of which are herein incorporated by reference.

### BACKGROUND OF THE INVENTION

1. Field of the Invention

The present invention relates to respiratory patient interface devices, and, in particular, to a respiratory patient interface device having a residual leak seal cushion.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion that rests beneath the patient's nose (such as a “pillows” style nasal cushion having nasal prongs that are received within the patient's nares or a “cradle” style nasal cushion that rests beneath and covers the patient's nares), a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

The mask component of a patient interface device has a certain amount of leak designed into it for the evaluation of safety and efficacy. This designed-in leak is known as intentional or exhalation leak, and is the leak used to verify mask safety. Residual or unintentional leak is the leak that is not designed into the mask but is still present due to assembly features, fit and/or manufacturing variation. Residual leak measurements have also been used to evaluate mask efficacy.

Numerous known nasal cushions integrally lock with mating semi-rigid and/or rigid structural housings or frames. These frames, which support the nasal cushions, normally include attachment features for enabling connection to a fluid coupling device such as a swivel style mask elbow or a ball-and-socket style mask elbow, which in turn are connected to the main gas delivery tube or conduit. In addition, most patient interface devices have features developed into their frames that are intended to reduce residual leak associated with the joint connection between the cushion and the frame. Such features, however, do not address any residual leak associated with the joint connection between a rigid structural housing/frame and elbow. Instead, current patient interface devices, with a rigid structural housing/frame, attempt to control and limit residual leak associated with the joint connection between the rigid structural housing/frame and elbow by requiring tight restrictions on part tolerances. Such tight restrictions, however, lead to higher manufacturing and assembly costs.

Furthermore, potential problems presented by uncontrolled residual leaks include lack of control of the direction of residual air flow, which may affect the patient and/or patient bed partner, lack of noise control, and ultimately patient compliance due to potential leak variations over the course of therapy.

### SUMMARY OF THE INVENTION

Accordingly, it is an object of the present invention to provide a patient interface device that overcomes the shortcomings of conventional patient interface devices that includes a sealing feature directed to controlling residual leak associated with the joint connection between the frame and elbow.

It is yet another object of the present invention to provide a method of delivering a flow of breathing gas that does not suffer from the disadvantages associated with conventional techniques. This object is achieved by providing a method that includes controlling residual leak associated with the joint connection between the frame and elbow.

In one embodiment, a patient interface device is provided that includes a fluid coupling device having an inlet end and an outlet end, a frame member having a connecting portion defining a first opening, and a cushion member coupled to the frame member. The cushion member has a main body portion and a coupling portion attached to the main body portion, the main body portion including an inwardly extending residual leak sealing member defining a second opening in the cushion member. The coupling portion is attached to a first side of the connecting portion in a manner wherein at least a portion of the residual leak sealing member extends over the first opening on the first side of the connecting portion around the entire periphery of the first opening, and the fluid coupling device is attached to a second side of the connecting portion opposite the first side in a manner wherein the outlet end of the fluid coupling device extends through the first opening and a terminal portion of the outlet end engages the residual leak sealing member without extending through the second opening and creates an airflow seal at a joint connection between the fluid coupling conduit and the frame member.

In another embodiment, a method of delivering a flow of breathing gas to a patient is provided that includes generating the flow of breathing gas, delivering the flow of breathing gas to a patient interface device, the patient interface device having a frame member having a connecting portion, a cushion member having a main body portion and a coupling portion coupled to a first side of the connecting portion, and a fluid coupling device coupled to a second side of the connecting portion opposite the first side, the flow being delivered to the fluid coupling device, and preventing residual leak flow from the patient interface device through a joint connection between the fluid coupling conduit and the frame member using a sealing member provided as part of the main body portion of the cushion member that creates a seal at the joint connection.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the present invention;

FIG. 2 is a front isometric view and FIG. 3 is a rear isometric view of a frame member forming a part of a patient interface device of the system of FIG. 1;

FIG. 4 is a side elevational view and FIG. 5 is a rear isometric view of a fluid coupling conduit 10 forming a part of a patient interface device of the system of FIG. 1;

FIG. 6 is a rear isometric view and FIG. 7 is a cross-sectional view of a cushion member forming a part of a patient interface device of the system of FIG. 1;

FIGS. 8 and 9 are cross-sectional views of a portion of the patient interface device of the system of FIG. 1;

FIG. 10 is a schematic diagram of a system adapted to provide a regimen of respiratory therapy to a patient according to an alternative exemplary embodiment of the present invention;

FIG. 11 is a rear isometric view and FIG. 12 is a cross-sectional view of an alternative cushion member forming a part of a patient interface device of the system of FIG. 10;

FIG. 13 is a rear isometric view and FIG. 14 is a cross-sectional view of an alternative cushion member that may form a part of the patient interface device of the system of FIGS. 11; and

FIG. 15 is a rear isometric view and FIG. 16 is a cross-sectional view of an alternative cushion member that may form a part of the patient interface device of the system of FIG. 10.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the, elements shown in the drawings and are not limiting upon the claims unless expressly recited therein.

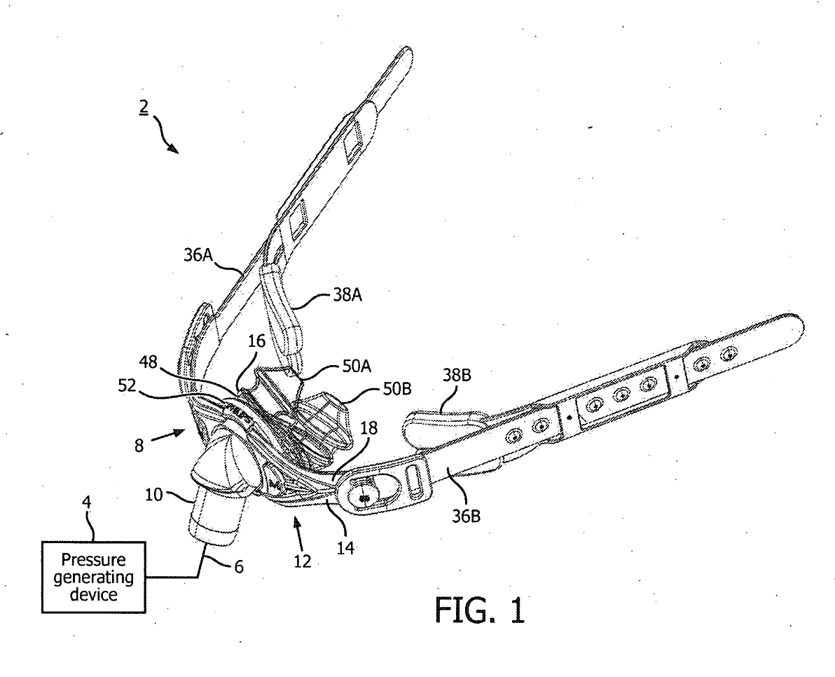

A system 2 adapted to provide a regimen of respiratory therapy to a patient according to one exemplary embodiment is generally shown in FIG. 1. System 2 includes a pressure generating device 4, a delivery conduit 6, and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

In the exemplary embodiment, patient interface 8 includes a patient sealing assembly 12, which in the illustrated embodiment is an alternative nasal mask. However, other types of patient sealing assemblies, such as, without limitation, a nasal/oral mask or a full face mask, which facilitates the delivery of the flow of breathing gas to the airway of a patient, may be substituted for patient sealing assembly 12 while remaining within the scope of the present invention. Patient sealing assembly 12 includes a frame member 14 having a cushion member 16 coupled thereto, each of which is described in greater detail below.

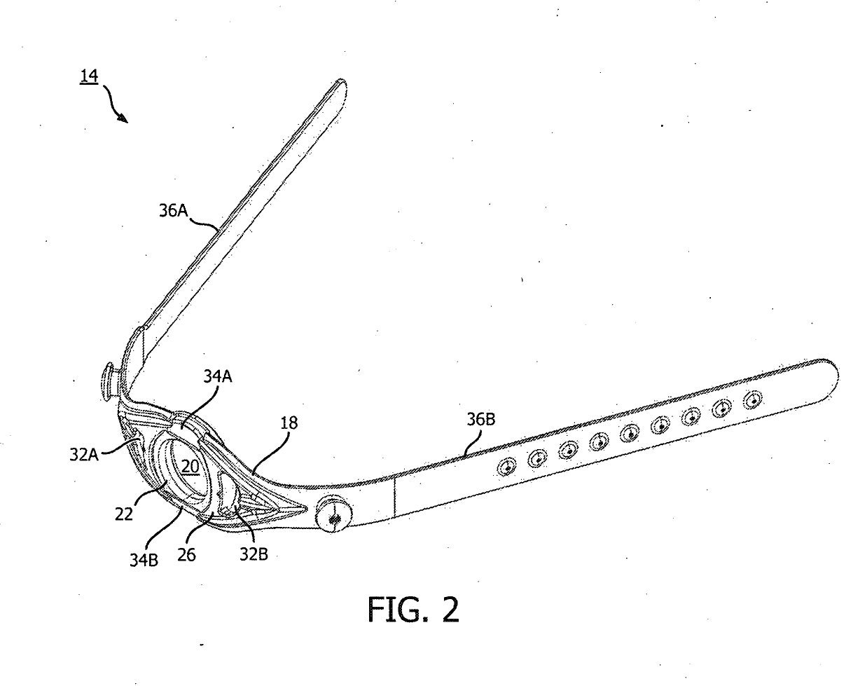

FIG. 2 is a front isometric view and FIG. 3 is a rear isometric view of frame member 14 according to the exemplary embodiment. In the illustrated embodiment, frame member 14 is made of a rigid or semi-rigid material, such as, without limitation, an injection molded thermoplastic or silicone. Frame member 14 includes a connecting portion 18 having a central annular opening 20 bounded by internal engagement surface 22. Connecting portion 18 further includes a cushion side 24 and a conduit side 26 opposite cushion side 24. As seen in FIG. 3, cushion side 24 includes an internal annular ridge member 28 and an external annular ridge member 29 each extending from cushion side 24 and which define between them an annular groove 30. In addition, connecting portion 18 includes openings 32A and 32B and slots 34A and 34B positioned around central annular opening 20. The function of each of the above described portions of frame member 14 is described in greater detail below. Frame member 14 further includes arms 36A and 36B which extend outwardly from connecting portion 18. As seen in FIG. 1, arms 36A and 36B each support an adjustable cheek support member 38A, 38B.

As also seen in FIG. 1, and as described in greater detail elsewhere herein, both fluid coupling conduit 10 and cushion member 16 are received by and supported by connecting portion 18. More specifically, fluid coupling conduit 10 is coupled to conduit side 26 of connecting portion 18 through opening 20, and cushion member 16 is coupled to cushion side 24 of connecting portion 18 in a manner which surrounds opening 20. This configuration allows the flow of breathing gas from pressure generating device 4 to be communicated to cushion member 16, and then to the airway of a patient.

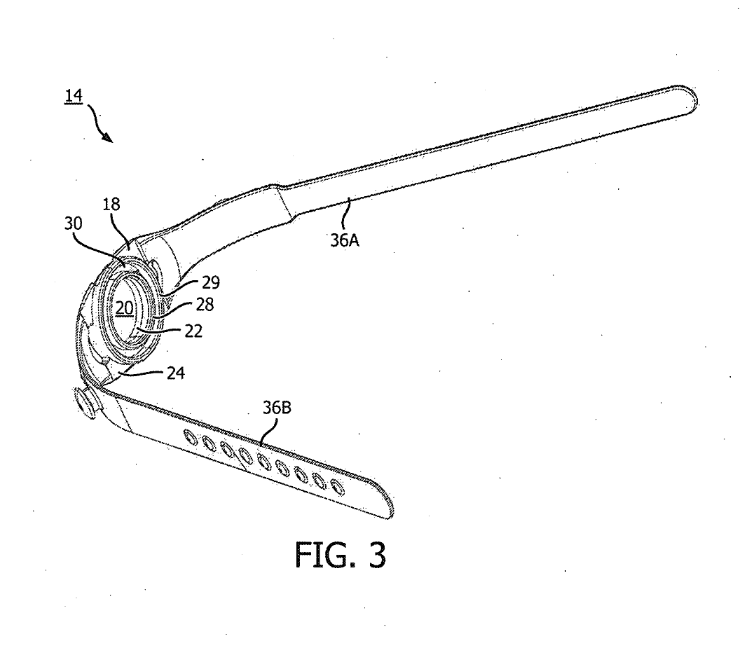

FIG. 4 is a side elevational view and FIG. 5 is a rear isometric view of fluid coupling conduit 10 according to the exemplary embodiment of the present invention. Referring to FIGS. 4 and 5, fluid coupling conduit 10, which is an elbow conduit, includes an inlet end 40 structured to be connected to delivery conduit 6 which in turn is connected to pressure generating device 4. Fluid coupling conduit 10 also includes an outlet end 42 having a cylindrical connector member 44 terminating in an end flange member 46. Outlet end 40 is structured to be removeably and sealingly coupled to connecting portion 18 in a manner described in greater detail herein.

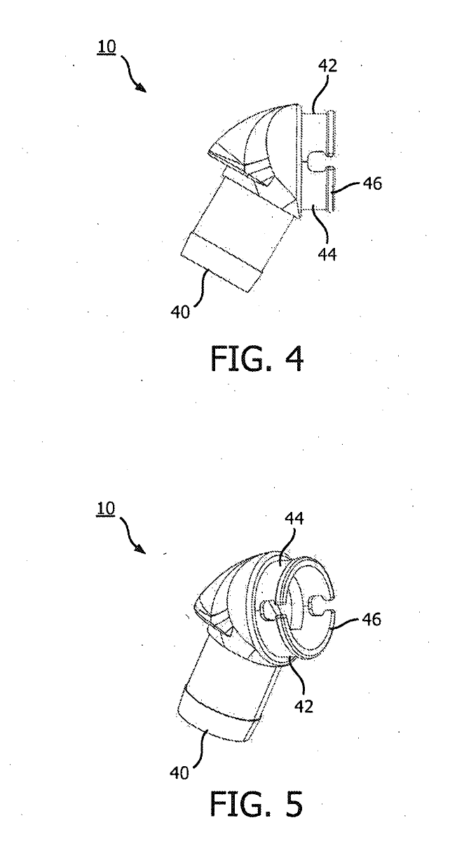

FIG. 6 is a rear isometric view and FIG. 7 is a cross-sectional view of cushion member 16. In the illustrated embodiment, cushion member 16 is a pillows style nasal cushion. Cushion member 16 is made of flexible, cushiony, elastomeric material, such as, without limitation, silicone, an appropriately soft thermoplastic elastomer, a closed cell foam, or any combination of such materials. Cushion member 16 includes a main body portion 48 having nasal prongs 50A and 50B (also referred to as pillows) extending from a top side thereof. In addition, cushion member 16 further includes a coupling portion 52 coupled to a bottom side of main body portion 48. Coupling portion 52 includes an internal annular ridge member 54 having tabs 56A, 56B extending therefrom, and an external annular ridge member 58 having tabs 60A, 60B, 60C, 60D extending therefrom. Internal annular ridge member 54 and external annular ridge member 58 define between them an annular groove 62. In addition, main body portion 48 includes an annular residual leak sealing member 64 extending inwardly from the base of internal annular ridge member 54 and the bottom of the main body portion 48. The function of the residual leak sealing member 64 is described in detail below.

In the illustrated embodiment, cushion member 16 is integrally molded from different durometer materials. In particular, main body portion 48, nasal prongs 50A and 50B, and residual leak sealing member 64 have a first durometer, and coupling portion 52 has a second durometer, wherein the first durometer is lower (softer) than the second durometer. In one particular, non-limiting embodiment, the first durometer is between 5 and 50 Shore A, and the second durometer is between 30 and 85 Shore A.

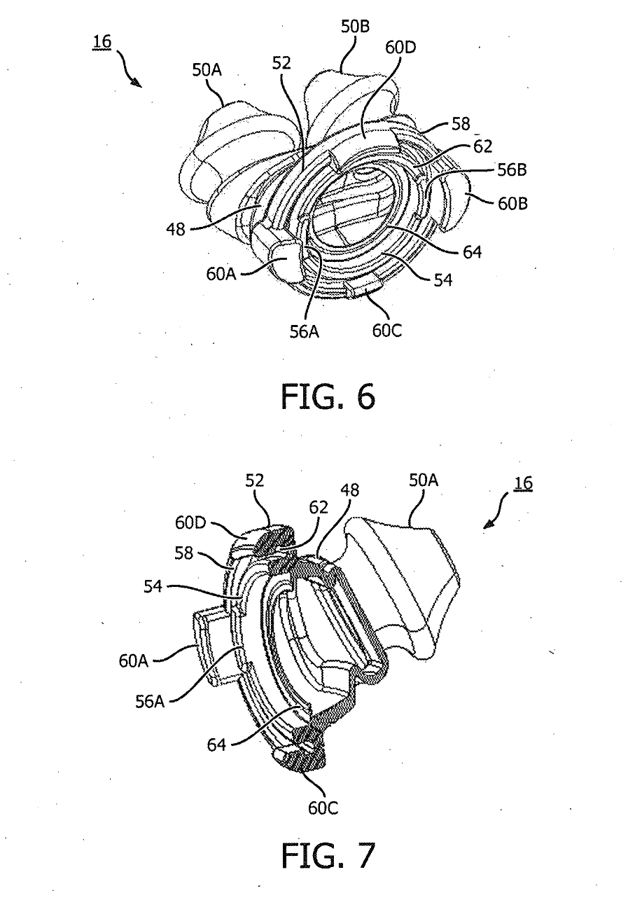

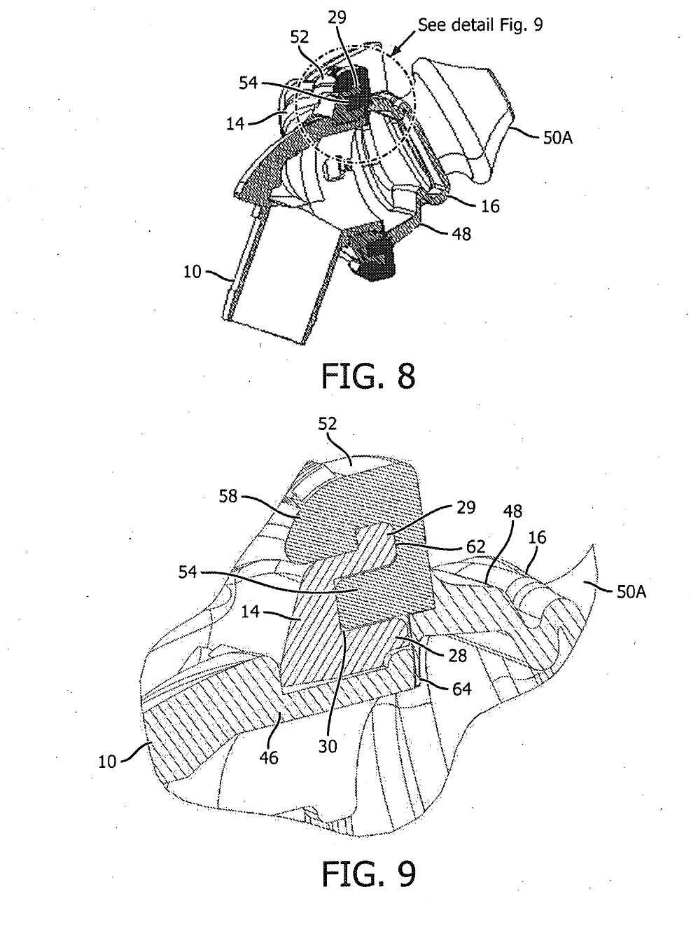

Referring to FIGS. 8 and 9, the assembly of patient interface device 8 will now be described. First, cushion member 16 is coupled to frame member 14 in a manner wherein internal annular ridge member 28 and external annular ridge member 29 of frame member 14 are mated with internal annular ridge member 54 and external annular ridge member 58 of cushion member 16. As seen in FIGS. 8 and 9, when this is done, internal annular ridge member 54 is received within groove 30 and external annular ridge member 29 is received within groove 62. In addition, as seen in FIG. 1, tabs 60A and 60B are received in openings 32A and 32B and tabs 60C and 60D are received in slots 34A and 34B. Furthermore, when so assembled, at least a portion of residual leak sealing member 64 protrudes into the airflow channel defined by opening 20. Next, fluid coupling conduit is 10 is inserted in frame member 14 through opening 20 until it butts up against and engages the portion of residual leak sealing member 64 protruding into the airflow channel as shown in FIGS. 8 and 9. In this manner, a fluid seal is created that restricts airflow from escaping through the junction of frame member 14 and fluid coupling conduit 10. In the exemplary embodiment, fluid coupling conduit 10 is held in place within frame member 14 by a friction and/or snap fit.

Thus, the present invention provides a configuration which enables better control over residual leak. This in turn enables a more scientific approach in designing mask leak for the purpose of efficiently evacuating enough CO2 to prevent re-breathing and in determining mask efficacy. Other benefits of the better control over residual leak provided by the present invention include less stringent design parameters on certain key features (lowering manufacturing and assembly costs), noise control and a higher overall patient satisfaction with the patient interface device 8 itself.

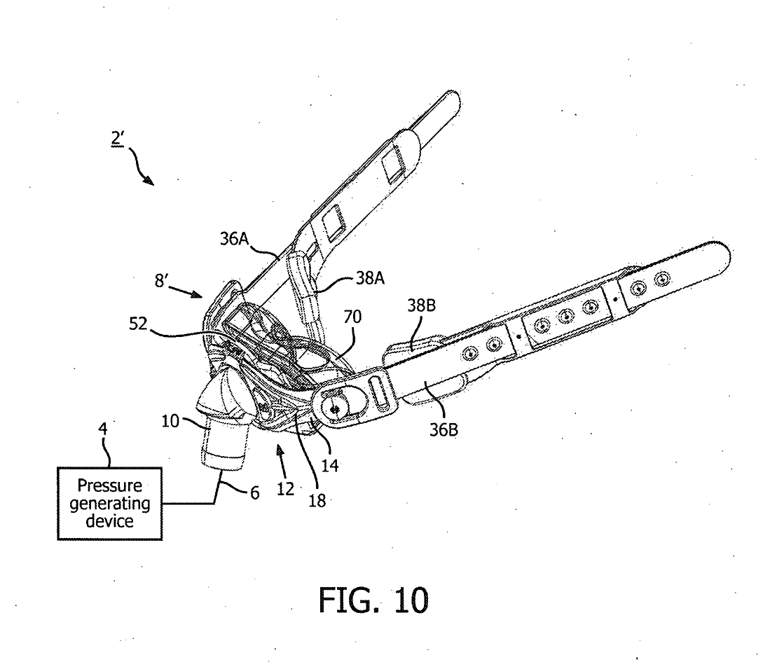

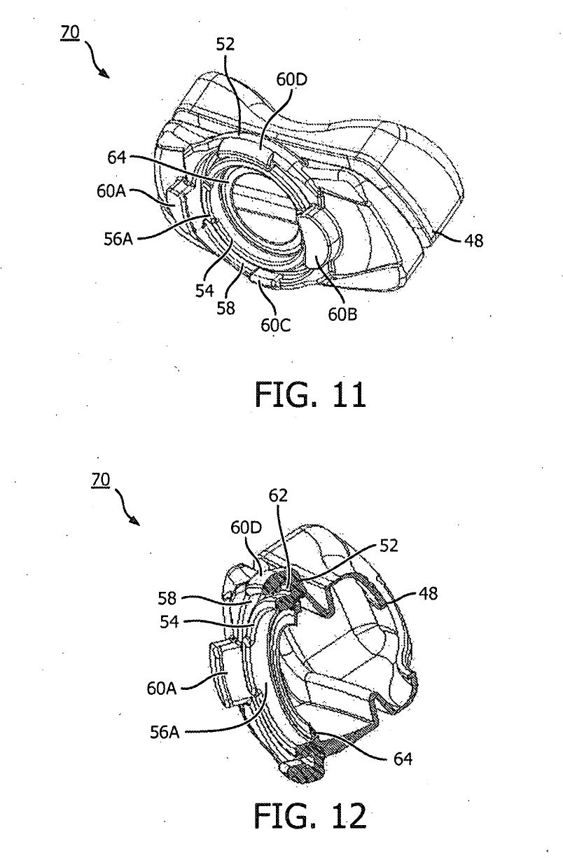

FIG. 10 is a schematic diagram of a system 2′ adapted to provide a regimen of respiratory therapy to a patient according to an alternative exemplary embodiment of the present invention. System 2′ includes many of the same components as system 2 described elsewhere herein, and like components are labeled with like reference numerals. System 2′ includes an alternative patient interface device 8′ that includes an alternative cushion member 70. Cushion member 70 is a cradle style nasal cushion. As seen in FIGS. 11 and 12, cushion member 70 includes many of the same components as cushion member 16, including residual leak sealing member 64, that enable it to provide the residual leak sealing functionality described in detail above.

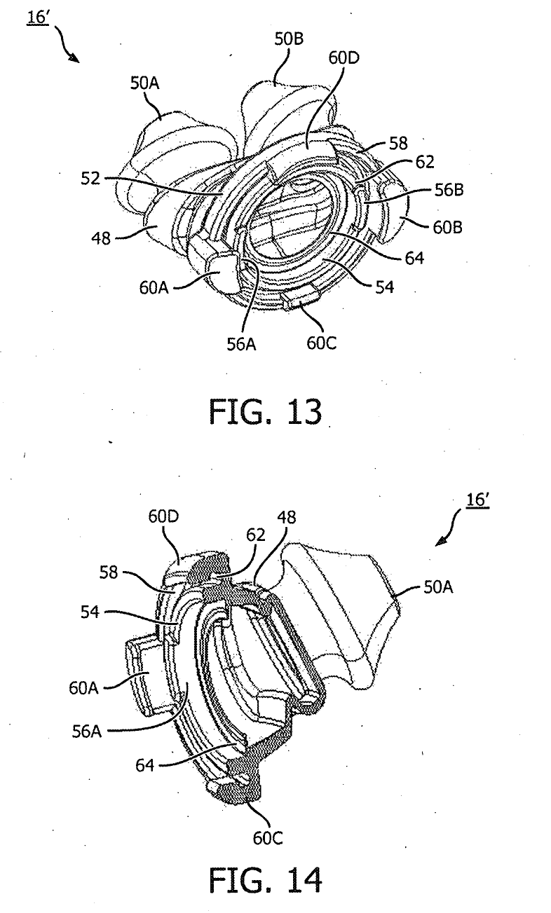

FIG. 13 is a rear isometric view and FIG. 14 is a cross-sectional view of an alternative pillows style cushion member 16′ that may be used in patent interface device 8 in place of cushion member 16. Cushion member 16′ is similar to cushion member 16 and includes many of the same components as cushion member 16, including residual leak sealing member 64, that enable it to provide the residual leak sealing functionality described in detail above. Cushion member 16′, however, unlike cushion member 16, is integrally molded of a single durometer material.

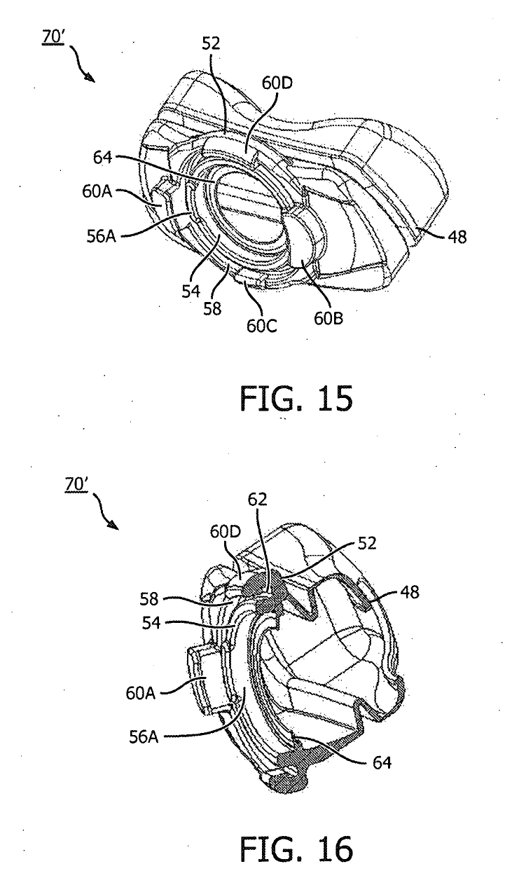

FIG. 15 is a rear isometric view and FIG. 16 is a cross-sectional view of an alternative cradle style cushion member 70′ that may be used in patent interface device 8′ in place of cushion member 70. Cushion member 70′ is similar to cushion member 70 and includes many of the same components as cushion member 70, including residual leak sealing member 64, that enable it to provide the residual leak sealing functionality described in detail above. Cushion member 70′, however, like cushion member 16′ is integrally molded of a single durometer material.

It can thus be appreciated that the present invention provides a patient interface device that includes a sealing feature directed to controlling residual leak associated with the joint connection between the rigid structural housing/frame and elbow

In the claims, any reference signs placed between parentheses shall not be construed as limiting the claim. The word “comprising” or “including” does not exclude the presence of elements or steps other than those listed in a claim. In a device claim enumerating several means, several of these mean's may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a fluid coupling device having an inlet end and an outlet end;
a frame member having a connecting portion defining a first opening;
a cushion member coupled to the frame member, the cushion member having a main body portion and a coupling portion attached to the main body portion, the main body portion including an inwardly extending residual leak sealing member defining a second opening in the cushion member, wherein the coupling portion is attached to a first side of the connecting portion in a manner wherein at least a portion of the residual leak sealing member extends over the first opening on the first side of the connecting portion around the entire periphery of the first opening, and wherein the fluid coupling device is attached to a second side of the connecting portion opposite the first side in a manner wherein the outlet end of the fluid coupling device extends through the first opening and a terminal portion of the outlet end engages the residual leak sealing member without extending through the second opening and creates an airflow seal at a joint connection between the fluid coupling conduit and the frame member.

2. The patient interface device according to claim 1, wherein the main body portion of the cushion member including the residual leak sealing member is made of a material having a first durometer and the coupling portion of the cushion member is made of a material having a second durometer, wherein the second durometer is higher than the first durometer.

3. The patient interface device according to claim 2, wherein the first durometer is between 5 and 50 Shore A and the second durometer is between 30 and 85 Shore A.

4. The patient interface device according to claim 1, wherein the cushion member is a nasal cushion.

5. The patient interface device according to claim 4, wherein nasal cushion is a pillows style cushion member having nasal prongs or a cradle style cushion member.

6. The patient interface device according to claim 1, wherein the first opening of the connecting portion is circular, wherein the residual leak sealing member is annular and the second opening is circular, and wherein a diameter of the first opening is greater than a diameter of the second opening.

7. The patient interface device according to claim 1, wherein the first side of the connecting portion includes a first connection assembly including a first internal annular ridge member, a first external annular ridge member and a first annular groove, wherein the coupling portion includes a second connection assembly including a second internal annular ridge member, a second external annular ridge member and a second annular groove, and wherein the first connection assembly is mated with the second connection assembly.

8. The patient interface device according to claim 1, wherein the fluid coupling conduit is an elbow conduit.

9. A method of delivering a flow of breathing gas to a patient, comprising:
generating the flow of breathing gas;
delivering the flow of breathing gas to a patient interface device, the patient interface device having a frame member having a connecting portion, a cushion member having a main body portion and a coupling portion coupled to a first side of the connecting portion, and a fluid coupling device coupled to a second side of the connecting portion opposite the first side, the flow being delivered to the fluid coupling device; and
preventing residual leak flow from the patient interface device through a joint connection between the fluid coupling conduit and the frame member using a sealing member provided as part of the main body portion of the cushion member that creates a seal at the joint connection.

10. The method according to claim 9, wherein the fluid coupling device has an inlet end and an outlet end, wherein the connecting portion defines a first opening, wherein the sealing member comprises an inwardly extending sealing member defining a second opening in the cushion member, wherein the coupling portion is attached to the first side of the connecting portion in a manner wherein at least a portion of the sealing member extends over the first opening on the first side of the connecting portion around the entire periphery of the first opening, and wherein the fluid coupling device is attached to the second side of the connecting portion in a manner wherein the outlet end of the fluid coupling device extends through the first opening and a terminal portion of the outlet end engages the sealing member without extending through the second opening and creates an airflow seal at the joint connection between the fluid coupling conduit and the frame member.

11. The method according to claim 10, wherein the main body portion of the cushion member including the sealing member is made of a material having a first durometer and the coupling portion of the cushion member is made of a material having a second durometer, wherein the second durometer is higher than the first durometer.

12. The method according to claim 11, wherein the first durometer is between 5 and 50 Shore A and the second durometer is between 30 and 85 Shore A.

13. The method according to claim 9, wherein the cushion member is a nasal cushion.

14. The method according to claim 13, wherein nasal cushion is a pillows style cushion member having nasal prongs or a cradle style cushion member.

15. The method according to claim 10, wherein the first opening of the connecting portion is circular, wherein the sealing member is annular and the second opening is circular, and wherein a diameter of the first opening is greater than a diameter of the second opening.

16. The method according to claim 9, wherein the first side of the connecting portion includes a first connection assembly including a first internal annular ridge member, a first external annular ridge member and a first annular groove, wherein the coupling portion includes a second connection assembly including a second internal annular ridge member, a second external annular ridge member and a second annular groove, and wherein the first connection assembly is mated with the second connection assembly.

17. The method according to claim 9, wherein the fluid coupling conduit is an elbow conduit.
